(19) United States
(12) Patent Application Publication
(10) Pub. No.: US 2014/0323872 A1
(43) Pub. Date: Oct. 30, 2014

(54) SCAN CONVERTER AND ULTRASONIC DIAGNOSTIC APPARATUS AND METHOD CAPABLE OF REAL-TIME INTERPOLATION WITHOUT DIRECTIONAL DISTORTION

(71) Applicants: Minyoung Eom, Seoul (KR); Hyunsook Lee, Gyeonggi-do (KR)

(72) Inventors: Minyoung Eom, Seoul (KR); Hyunsook Lee, Gyeonggi-do (KR)

(73) Assignee: ALPINION MEDICAL SYSTEMS CO., LTD., Hwaseong-si, Gyeonggi-do (KR)

(21) Appl. No.: 14/371,586

(22) PCT Filed: Jan. 11, 2012
(86) PCT No.: PCT/KR2012/000265

§ 371 Date: Jul. 10, 2014

(30) Foreign Application Priority Data
Jan. 11, 2012 (KR) 10-2012-0003290

(51) Int. Cl. G01S 7/52; G06F 17/17; A61B 8/06

(52) U.S. Cl. CPC G01S 7/52044; A61B 8/06; G06F 17/17
USPC 600/459; 708/290

(57) ABSTRACT

The present disclosure in one or more embodiments provides a scan converter, an ultrasound diagnostic apparatus, and a method capable of performing real-time interpolation without directional distortion. An embodiment of the present disclosure prevents directional distortion of velocity data by performing vector interpolation by using a weight according to distances of four adjacent complex signals in color flow-mode (C-mode) and achieves real-time scan conversion by constructing a lookup table for vector interpolation.

### TECHNICAL FIELD

The present disclosure in one or more embodiments relates to a scan converter, an ultrasound diagnostic apparatus and a method thereof, which are capable of performing real-time interpolation without directional distortion. More particularly, the present disclosure relates to a scan converter, an ultrasound diagnostic apparatus and a method thereof, which comprise performing real-time interpolation without directional distortion in carrying out scan conversion of velocity data of blood flow in C-mode and a method for constructing a lookup table for the interpolation.

### BACKGROUND

The statements in this section merely provide background information related to the present disclosure and may not constitute prior art.

Ultrasound imaging apparatus employs a transducer transmitting ultrasound signal to a subject on its surface contacted by the transducer, and receives ultrasound signal reflected from the subject (hereinafter called reflection or echo signals), forms an ultrasound image of the subject based on the reflection signals received through the transducer, and displays the ultrasound image on a display. The ultrasound image may be expressed as brightness-mode (B-mode), color flow-mode (C-mode), BC-mode, etc. The B-mode is an image mode using a reflection coefficient which depends upon the difference in acoustic impedance between tissues and the C-mode is an image mode for displaying blood flow or motion of a subject by using Doppler effect. The BC-mode is an image mode providing both the B-mode image and the C-mode image.

Lacking velocity data of a subject, the B-mode may be free from a directional distortion, whereas the C-mode (including BC-mode as will be so described hereinafter) includes velocity data and the directional distortion may occur when using a conventional interpolation method.

### DISCLOSURE

### Technical Problem

Therefore, the present disclosure has been made partly in an effort to prevent the directional distortion of velocity data by performing vector interpolation by using four adjacent complex signals in consideration of a weight in the C-mode according to distance and to achieve real-time scan conversion by constructing a lookup table for vector interpolation.

### SUMMARY

In accordance with some embodiments of the present disclosure, a scan converter is provided for generating an interpolation signal by performing vector interpolation to represent, in Cartesian coordinates, a complex signal obtained in polar coordinates by a transducer. The scan converter comprises a complex signal selector, a first temporal interpolation signal generator, a second temporal interpolation signal generator and an interpolation signal generator. The complex signal selector is configured to select four complex signals adjacent to the interpolation signal. The first temporal interpolation signal generator is configured to generate four first temporal interpolation signals by vector-interpolating every two complex signals which are two adjacent interpolatable signals among the selected four complex signals by using a weight according to a distance between each of the two interpolatable signals and the interpolation signal. The second temporal interpolation signal generator is configured to generate two second temporal interpolation signals by vector-interpolating every two first temporal interpolation signals which are two opposite interpolatable signals centering on the interpolation signal among the generated four first temporal interpolation signals by using a weight according to a distance between each of the two interpolatable signals and the interpolation signal. And the interpolation signal generator is configured to generate the interpolation signal by vector-interpolating by using an averaged weight from averaging the generated two second temporal interpolation signals subject to interpolation as the two opposite interpolatable signals.

Here, the complex signal selector may make selections of two scan lines as a first scan line and a second scan line adjacent to the interpolation signal among multiple scan lines in radial directions, two complex signals which are adjacent to the interpolation signal and are located on the first scan line, and two complex signals which are adjacent to the interpolation signal and are located on the second scan line.

The weight according to the distance for the first temporal interpolation signal generator may be a ratio of a distance between one of the adjacent interpolatable signals and the interpolation signal to a sum of the distance between the one interpolatable signal and the interpolation signal and a distance between the other of the adjacent interpolatable signals and the interpolation signal.

The weight according to distance for the second temporal interpolation signal generator may be a ratio of a distance between one of the opposite interpolatable signals and the interpolation signal to a sum of the distance between the one interpolatable signal and the interpolation signal and a distance between the other of the interpolatable signals and the interpolation signal.

The averaged weight may be 0.5.

The interpolation signal may include distance information obtained through a geometric mean of distances of the two second temporal interpolation signals and phase information obtained through an arithmetic mean of phases of the two second temporal interpolation signals.

The scan converter may further comprise an interpolation phase information extractor configured to extract phase information θ₃ of the first and second temporal interpolation signals and of the interpolation signal from using a lookup table. Here, the lookup table may store the phase information θ₃ predetermined according to phase magnitudes θ₁ and θ₂ of the two interpolatable signals and weights α and β depending on the respective distances from the two interpolatable signals to the interpolation signal.

The lookup table may store the phase information expressed by

$${θ}_{3} = α {θ}_{1} + β {θ}_{2} = {tan}^{- 1} ( \frac{sin ( α {θ}_{1} + β {θ}_{2} )}{cos ( {αθ}_{1} + {βθ}_{2} )} )$$

where α+β=1.

Another aspect of the present disclosure provides a vector interpolation method for generating an interpolation signal by performing vector interpolation to enable a scan converter to represent, in Cartesian coordinates, a complex signal obtained in polar coordinates by a transducer. The method comprises selecting four complex signals adjacent to the interpolation signal; generating four first temporal interpolation signals by vector-interpolating every two complex signals which are two adjacent interpolatable signals among the selected four complex signals by using a weight according to a distance between each of the two interpolatable signals and the interpolation signal; generating two second temporal interpolation signals by vector-interpolating every two first temporal interpolation signals which are two opposite interpolatable signals centering on the interpolation signal among the generated four first temporal interpolation signals by using a weight according to a distance between each of the two interpolatable signals and the interpolation signal; and generating the interpolation signal by vector-interpolating by using an averaged weight from averaging the generated two second temporal interpolation signals subject to interpolation as the two opposite interpolatable signals.

Here, the averaged weight may be 0.5.

The vector interpolation method may further comprise extracting phase information θ₃ of the first and second temporal interpolation signals and of the interpolation signal from using a lookup table. Here, the lookup table may store the phase information θ₃ predetermined according to phase magnitudes θ₁ and θ₂ of the two interpolatable signals and weights α and β depending on the respective distances from the two interpolatable signals to the interpolation signal.

In accordance with yet another aspect of the present disclosure, an ultrasound diagnostic apparatus comprises a transducer, a beamformer, a demodulator and a scan converter. The transducer is configured to convert an electric analog signal into an ultrasound signal, transmit the ultrasound signal to a subject, and convert an ultrasound signal reflected from the subject into an electric analog signal. The beamformer is configured to form a receive-focusing signal based on the converted electric analog signal. The demodulator is configured to form I data comprising in-phase components and Q data comprising quadrature-phase components by demodulating the receive-focusing signal into a baseband signal. And the scan converter is configured to generate an interpolation signal by performing vector interpolation to represent, in Cartesian coordinates, a complex signal obtained in polar coordinates, based on the I data and the Q data. Here, the scan converter generates the interpolation signal by vector-interpolating four complex signals adjacent to the interpolation signal by using a weight according to a distance between each of the four complex signals and the interpolation signal.

The ultrasound diagnostic apparatus may further comprises an analog/digital (A/D) converter configured to convert the electric analog signal transformed by the transducer into an electric digital signal.

The scan converter may comprise a complex signal selector, a first temporal interpolation signal generator, a second temporal interpolation signal generator and an interpolation signal generator. The complex signal selector is configured to select four complex signals adjacent to the interpolation signal. The first temporal interpolation signal generator is configured to generate four first temporal interpolation signals by vector-interpolating every two complex signals which are two adjacent interpolatable signals among the selected four complex signals by using a weight according to a distance between each of the two interpolatable signals and the interpolation signal. The second temporal interpolation signal generator is configured to generate two second temporal interpolation signals by vector-interpolating every two first temporal interpolation signals which are two opposite interpolatable signals centering on the interpolation signal among the generated four first temporal interpolation signals by using a weight according to a distance between each of the two interpolatable signals and the interpolation signal. And the interpolation signal generator is configured to generate the interpolation signal by vector-interpolating by using an averaged weight from averaging the generated two second temporal interpolation signals subject to interpolation as the two opposite interpolatable signals.

Here, the averaged weight may be 0.5.

The scan converter may further comprise an interpolation phase information extractor configured to extract phase information θ₃ of the first and second temporal interpolation signals and of the interpolation signal from using a lookup table. Here, the lookup table may store the phase information θ₃ predetermined according to phase magnitudes θ₁ and θ₂ of the two interpolatable signals and weights α and β depending on the respective distances from the interpolatable signals to the interpolation signal.

### ADVANTAGEOUS EFFECTS

According to the present disclosure as described above, the directional distortion of velocity data can be prevented by performing vector interpolation by using four adjacent complex signals in the C-mode in consideration of a weight according to distance, and real-time scan conversion can be achieved by constructing a lookup table for vector interpolation.

### DESCRIPTION OF DRAWINGS

FIG. 1 is a schematic diagram of an ultrasound imaging diagnostic apparatus 100 according to some embodiments of the present disclosure.

FIG. 2 is a schematic diagram of a scan converter 150 according to some embodiments of the present disclosure.

FIG. 3 is a diagram of a process of selecting four complex signals adjacent to an interpolation signal Z_{NEW} in Cartesian coordinates in a vector interpolation method performed by the scan converter 150.

FIG. 4 is a diagram of a detailed vector interpolation method performed by the scan converter 150 using four selected complex signals.

FIG. 5 is a diagram of a lookup table construction method for vector interpolation for real-time implementation.

### DETAILED DESCRIPTION

Hereinafter, at least one embodiment of the present disclosure will be described in detail with reference to the accompanying drawings.

In the following description, like reference numerals designate like elements although the elements are shown in different drawings. Further, in the following description of the at least one embodiment, a detailed description of known functions and configurations incorporated herein will be omitted for the purpose of clarity and for brevity.

Additionally, in describing the components of the present disclosure, terms like first, second, A, B, (a), and (b) are used. These are solely for the purpose of differentiating one component from another, and one of ordinary skill would understand that the terms are not to imply or suggest the substances, order or sequence of the components. If a component is described as ‘connected’, ‘coupled’, or ‘linked’ to another component, one of ordinary skill in the art would understand that the components are not necessarily directly ‘connected’, ‘coupled’, or ‘linked’ but also are indirectly ‘connected’, ‘coupled’, or ‘linked’ via a third component.

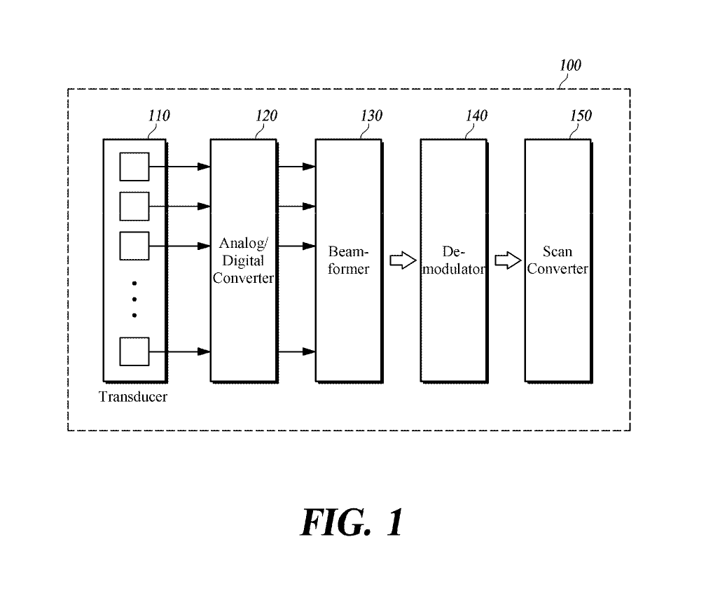

FIG. 1 is a schematic diagram of the ultrasound diagnostic apparatus 100 according to some embodiments of the present disclosure. The ultrasound diagnostic apparatus 100 performs vector interpolation for the C-mode image output. The C-mode refers to an image mode for displaying blood flow or motion of an object, using Doppler effect.

Referring to FIG. 1, the ultrasound diagnostic apparatus 100 according to some embodiments of the present disclosure may include a transducer 110, an analog/digital (A/D) converter 120, a beamformer 130, a demodulator 140 and a scan converter 150.

The transducer 110 converts an electric analog signal into an ultrasound signal, transmits the ultrasound signal to a subject, and converts ultrasound signals reflected from the subject (hereinafter, echo signals) into an electric analog signal. The transducer 110 typically includes a plurality of transducer elements coupled to each other. The present disclosure relates to a vector interpolation based on a C-mode image and, therefore, a description thereof will be given by focusing on vector interpolation based on the C-mode image. The ultrasound signal transmitted by the transducer 110 is scattered by a moving subject and forms echo signals. In other words, the ultrasound signal transmitted by the transducer 110 is scattered by the flow of red blood cells in blood vessels and forms the echo signals. Since the subject is in motion, a frequency of the echo signal is different from a frequency of the transmitted ultrasound signal due to Doppler effect. The difference between the frequency of the transmitted ultrasound signal and the frequency of the echo signal is called Doppler frequency which is proportional to the velocity of the subject. Accordingly, the moving direction and velocity of the subject can be detected by using the Doppler frequency.

The A/D (analog/digital) converter 120 converts the electric analog signal converted by the transducer 110 into an electric digital signal. Although FIG. 1 illustrates the A/D converter 120 as positioned between the transducer 110 and the beamformer 130, the position of the A/D converter 120 is not limited thereto and the A/D converter may be located at any part of the ultrasound diagnostic apparatus 100.

The beamformer 130 forms a receive-focusing signal based on the electric digital signal converted by the A/D converter 120. In other words, the beamformer 130 applies proper delays on each electric digital signal in consideration of time during which the signal reaches each transducer element of the transducer 110 from the subject and adds up the delayed electric digital signals, thereby forming the receive-focusing signal.

The demodulator 140 demodulates the receive-focusing signal generated by the beamformer 130 to baseband and thereby forms I data comprising in-phase components and Q data comprising quadrature-phase components. The demodulator 140 first passes the receive-focusing signal generated by the beamformer 130 through a high-pass filter, multiplies the high-pass filtered signal by a cosine function and a sine function, and then passes the multiplied signal through a low-pass filter, thereby forming the I data and Q data demodulated to baseband.

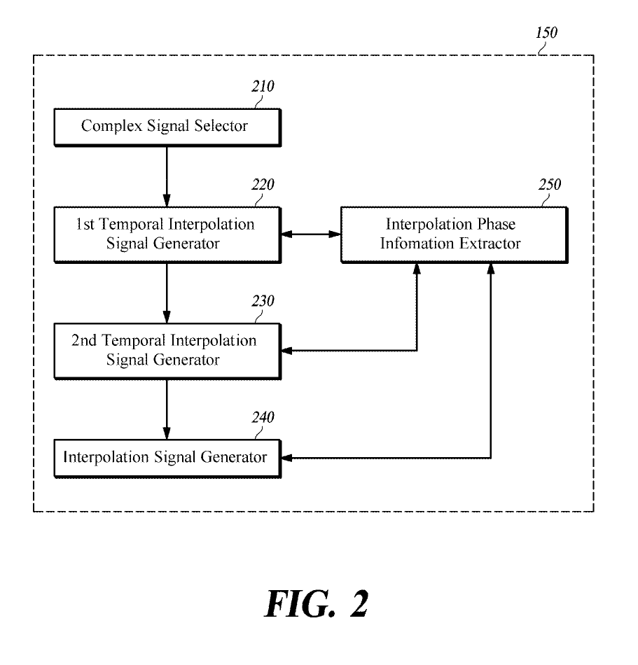

The scan converter 150 carries out a vector interpolation on a complex signal which is obtained based on the I data and the Q data generated by the demodulator 140 and is present on a scan line in a radial direction as well in polar coordinates, in order to generate an interpolation signal Z_{NEW} for representing the complex signal in Cartesian coordinates. Since the complex signal appearing in polar coordinates is in a format that cannot be output on a display, the complex signal in polar coordinates is vector-interpolated to an interpolation signal in Cartesian coordinates, which is a form capable of being output by the scan converter 150. The interpolation signal refers to a complex signal for an arbitrary point to be vector-interpolated in Cartesian coordinates. A vector interpolation method performed by the scan converter 150 will be described with reference to accompanying FIGS. 2 to 5.

FIG. 2 is a schematic diagram of the scan converter 150 according to some embodiments of the present disclosure.

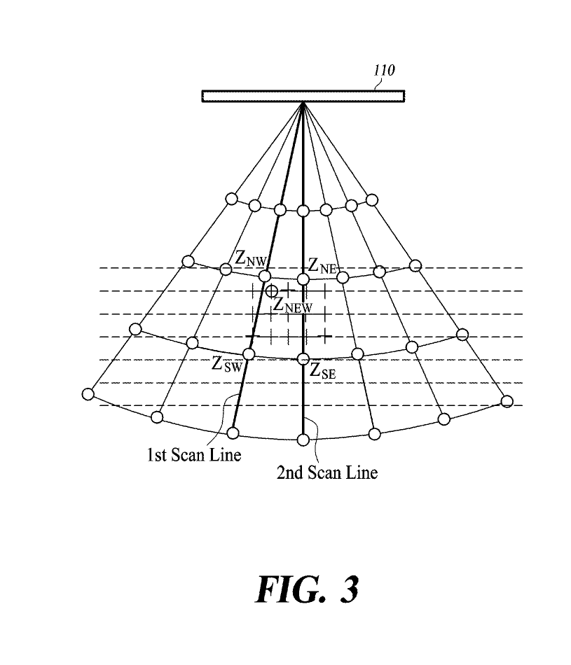

Referring to FIG. 2, the scan converter 150 according to some embodiments of the present disclosure may include a complex signal selector 210, a first temporal interpolation signal generator 220, a second temporal interpolation signal generator 230, an interpolation signal generator 240 and an interpolation phase information extractor 250. The configuration of the scan converter 150 according to some embodiments of the present disclosure is described with reference to FIGS. 3 to 5.

FIG. 3 is a diagram of a process performed by the complex signal selector 210 for selecting four complex signals adjacent to an interpolation signal Z_{NEW} in Cartesian coordinates in the vector interpolation method performed by the scan converter 150. Interpolation signal Z_{NEW} refers to a complex signal for an arbitrary point to be vector-interpolated in Cartesian coordinates.

The vector interpolation method performed by the scan converter 150 will now be described with reference to FIG. 3. The complex signal selector 210 selects two scan lines (first and second lines) adjacent to the interpolation signal Z_{NEW} in Cartesian coordinates from a plurality of scan lines in different radial directions. Once the two first and second scan lines are selected, the scan converter 150 selects four complex signals on the two scan lines, which are adjacent to interpolation signal Z_{NEW}. The scan converter 150 selects a complex signal Z_{NW} which is adjacent to interpolation signal Z_{NEW} and is located at one side of the first scan line, a complex signal Z_{SW} which is adjacent to interpolation signal Z_{NEW} and is located at the other side of the first scan line, a complex signal Z_{NE} which is adjacent to interpolation signal Z_{NEW} and is located at one side of the second scan line, and a complex signal Z_{SE} which is adjacent to interpolation signal Z_{NEW} and is located at the other side of the second scan line.

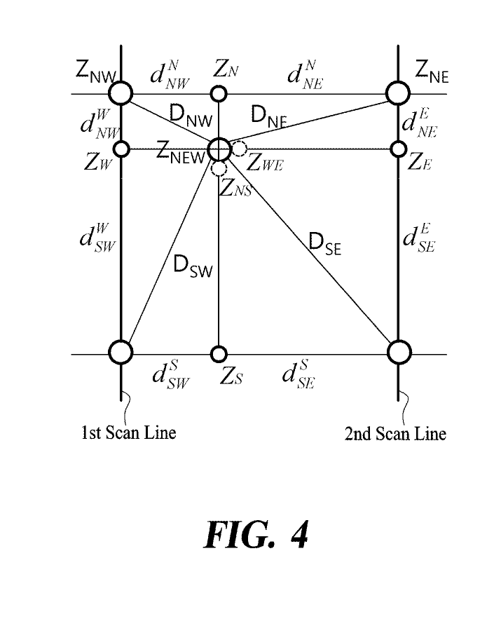

FIG. 4 is a diagram of a detailed vector interpolation method performed by the scan converter 150 using the four selected complex signals.

A method for performing vector interpolation upon interpolation signal Z_{NEW} in Cartesian coordinates, using four complex signals Z_{NW}, Z_{NE}, Z_{SW} and Z_{SE} will now be described with reference to FIG. 4. Interpolation signal Z_{NEW} refers to a complex signal for an arbitrary point to be vector-interpolated in Cartesian coordinates.

As a preliminary, a typical vector interpolation method is explained by using two complex signals Z_{A} and Z_{B}. Vector interpolation is performed by applying weights α and β according to distance with respect to an arbitrary point Z_{C}, as indicated in Equation 1.

z_{A}=r_{A}e^{jθ}^{_{A}}, z_{B}=r_{B}e^{jθ}^{_{B}}

z_{C}=z_{A}^{α}z_{B}^{β}=r_{A}^{α}r_{B}^{β}e^{j(αβ}^{_{A}}^{+βθ}^{_{B}}⁾, α+β=1 Equation 1

Vector interpolation will be described below based on Equation 1.

With reference to FIG. 4, a method of the first temporal interpolation signal generator 220 will be described for calculating weights α and β according to distance between each of two interpolatable signals and interpolation signal Z_{NEW} with respect to every two complex signals which are two adjacent interpolatable signals among four selected complex signals Z_{NW}, Z_{NE}, Z_{SW} and Z_{SE}. Each of distance dependent weights α and β is the ratio of the distance between one interpolatable signal and interpolation signal Z_{NEW} to the sum of the distance between the one interpolatable signal and interpolation signal Z_{NEW} and the distance between the other interpolatable signal and interpolation signal Z_{NEW}. Distance D_{NW} between interpolation signal Z_{NEW} and signal Z_{NW} is known, and distance D_{NE} between interpolation signal Z_{NEW} and signal Z_{NE}, distance D_{SW} between interpolation signal Z_{NEW} and signal Z_{SW}, and distance D_{SE} between interpolation signal Z_{NEW} and signal Z_{SE} are known as well. Accordingly, the distance dependent weights can be calculated as indicated in Equation 2.

$${d}_{NW}^{W} = \frac{{D}_{NW}}{{D}_{NW} + {D}_{SW}} {d}_{SW}^{W} = \frac{{D}_{SW}}{{D}_{NW} + {D}_{SW}} \\ {d}_{SW}^{S} = \frac{{D}_{SW}}{{D}_{SW} + {D}_{Se}} {d}_{SE}^{S} = \frac{{D}_{SE}}{{D}_{SW} + {D}_{SE}} \\ {d}_{SE}^{E} = \frac{{D}_{SE}}{{D}_{SE} + {D}_{NE}} {d}_{SW}^{E} = \frac{{D}_{NE}}{{D}_{SE} + {D}_{NE}} \\ {d}_{NW}^{N} = \frac{{D}_{NW}}{{D}_{NW} + {D}_{NE}} {d}_{NE}^{N} = \frac{{D}_{NE}}{{D}_{NW} + {D}_{NE}} \qquad Equation 2$$

A method of the first temporal interpolation signal generator 220 is described with reference to FIG. 4 for generating four first temporal interpolation signals Z_{W}, Z_{E}, Z_{N} and Z_{S} by vector-interpolating every two complex signals which are two adjacent interpolatable signals among four selected complex signals Z_{NW}, Z_{NE}, Z_{SW} and Z_{SE}, using weights according to distances from two respective interpolatable signals to interpolation signal Z_{NEW}.

First temporal interpolation signal Z_{W} is generated by vector-interpolating two interpolatable signals Z_{NW} and Z_{SW}, using weights according to distances from interpolatable signals Z_{NW} and Z_{SW} to interpolation signal Z_{NEW} as in Equation 3.

Z_{W}=Z_{SW}^{d}^{_{SW}}^{^{W}}·Z_{NW}^{d}^{_{SW}}^{^{W}} Equation 3

First temporal interpolation signal Z_{E} is generated by vector-interpolating two interpolatable signals Z_{NE} and Z_{SE}, using weights according to distances from interpolatable signals Z_{NE} and Z_{SE} to interpolation signal Z_{NEW} as in Equation 4.

Z_{E}=Z_{SE}^{d}^{_{NE}}^{^{E}}·Z_{NE}^{d}^{_{SE}}^{^{E}} Equation 4

First temporal interpolation signal Z_{N} is generated by vector-interpolating two interpolatable signals Z_{NW} and Z_{NE}, using weights according to distances from interpolatable signals Z_{NW} and Z_{NE} to interpolation signal Z_{NEW} as in Equation 5.

Z_{N}=Z_{NW}^{d}^{_{NE}}^{^{N}}·Z_{NE}^{d}^{_{NE}}^{N} Equation 5

First temporal interpolation signal Z_{S} is generated by vector-interpolating two interpolatable signals Z_{SW} and Z_{SE}, using weights according to distances from interpolatable signals Z_{SW} and Z_{SE} to interpolation signal Z_{NEW} as indicated in FIG. 6.

Z_{S}=Z_{SW}^{d}^{_{SE}}^{^{S}}·Z_{SE}^{d}^{_{SW}}^{^{S}} Equation 6

With reference to FIG. 4, a method will now be described in which the second temporal interpolation signal generator 230 generates two second temporal interpolation signals Z_{WE} and Z_{NS} by vector-interpolating every two first temporal interpolation signals which are two opposite interpolatable signals centering on interpolation signal Z_{NEW}, among the four generated first temporal interpolation signals Z_{W}, Z_{E}, Z_{N} and Z_{S} by using weights according to distances from the two respective interpolatable signals to interpolation signal Z_{NEW}. The distance dependent weight is the ratio of the distance between one interpolatable signal and interpolation signal Z_{NEW} to the sum of the distance between the one interpolatable signal and interpolation signal Z_{NEW} and the distance between the other interpolatable signal and interpolation signal Z_{NEW}.

The second temporal interpolation signal Z_{WE} is generated by vector-interpolating the two interpolatable signals Z_{W} and Z_{E} using weights according to distances from interpolatable signals Z_{W} and Z_{E} to interpolation signal Z_{NEW} as indicated in Equation 7. The position of Z_{WE} illustrated in FIG. 4 is just an example to simplify explanation and the accurate enumerative position of Z_{WE} may change.

Z_{WE}=Z_{W}^{d}^{_{NE}}^{^{N}}·Z_{E}^{d}^{_{NW}}^{^{N}}=Z_{W}^{d}^{_{SE}}^{^{S}}·Z_{E}^{d}^{_{SW}}^{^{S}} Equation 7

The second temporal interpolation signal Z_{NS} is generated by vector-interpolating the two interpolatable signals Z_{N} and Z_{S} using weights according to distances from interpolatable signals Z_{N} and Z_{S} to interpolation signal Z_{NEW} as indicated in Equation 8. The position of Z_{NS} illustrated in FIG. 4 is just an example to simplify explanation and an accurate enumerative position of Z_{NS} may change.

Z_{NS}=Z_{N}^{d}^{_{SW}}^{^{W}}·Z_{S}^{d}^{_{NW}}^{^{W}}=Z_{W}^{d}^{_{SE}}^{^{E}}·Z_{S}^{d}^{_{NE}}^{^{E}} Equation 8

With reference to FIG. 4, a method of the interpolation signal generator 240 is described for generating interpolation signal Z_{NEW} by vector-interpolating by using a weight from averaging the generated two second temporal interpolation signals Z_{WE} and Z_{NS} subject to interpolation as two interpolatable signals.

Interpolation signal Z_{NEW} is generated by vector-interpolating the two interpolatable signals Z_{WE} and Z_{NS} using weight 0.5 from averaging interpolatable signals Z_{WE} and Z_{NS}. In other words, vector-interpolating with the averaged weight of 0.5 provides distance information of interpolation signal Z_{NEW} vector-interpolated by a geometric mean of the distance of Z_{WE} and the distance of Z_{NS} and provides phase information of interpolation signal Z_{NEW} by an arithmetic mean of the phase of Z_{WE} and the phase of Z_{NS}.

$${Z}_{NEW} = {Z}_{WE}^{0.5} \cdot {Z}_{NS}^{0.5} = \sqrt{{r}_{WE} {r}_{NS}} {e}^{j ( \frac{{θ}_{WE} + {θ}_{NS}}{2} )} \qquad Equation 9$$

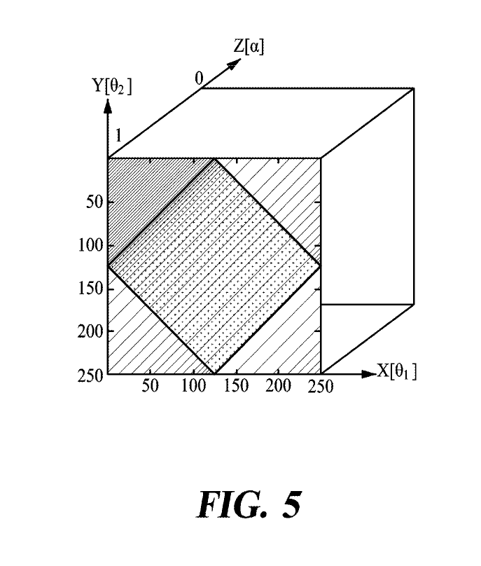

FIG. 5 is a diagram of a lookup table construction method for vector interpolation for real-time implementation thereof.

To obtain phase information θ₃ of the vector-interpolated first and second temporal interpolation signals Z_{W}, Z_{E}, Z_{N}, Z_{S}, Z_{WE} and Z_{NS} and of interpolation signal Z_{NEW}, the interpolation phase information extractor 250 may extract the same information from a lookup table which can be used to store θ₃ predetermined by phase magnitudes θ₁ and θ₂ of two interpolatable signals and weights α and β depending on the distances from the two respective interpolatable signals to interpolation signal Z_{NEW}. The first and second temporal interpolation signals Z_{W}, Z_{E}, Z_{N}, Z_{S}, Z_{WE} and Z_{NS} which are vector-interpolated by using two interpolatable signals and interpolation signal Z_{NEW} are respectively formed of distance information and phase information to readily permit the scan converter 150 to calculate the distance information, but a delay in calculation of the phase information θ₃ may adversely affect the real-time implementation of scan conversion. The real-time implementation can be achieved by extracting the phase information θ₃ from a generated lookup table.

A method of the interpolation phase information extractor 250 for calculating phase information θ₃ in order to construct the lookup table is expressed by Equation 10.

$${e}^{j ( α {θ}_{1} + β {θ}_{2} )} = cos ( α {θ}_{1} + {βθ}_{2} ) + j sin ( {αθ}_{1} + {βθ}_{2} ) \\ {θ}_{3} = α {θ}_{1} + {βθ}_{2} = {tan}^{- 1} ( \frac{sin ( α {θ}_{1} + {βθ}_{2} )}{cos ( {αθ}_{1} + {βθ}_{2} )} ) \\ α + β = 1 \qquad Equation 10$$

After obtaining phase information θ₃ by Equation 10, the interpolation phase information extractor 250 may construct the lookup table as illustrated in FIG. 5. Specifically, the lookup table can be generated by presenting approximately 256×256 block of phases θ₁ and θ₂ of the two interpolatable signals on the X-axis and Y-axis in the range of −π<0_{1,2}<π and by presenting a predetermined magnitude of one of weights α and β according to distances from the first and second interpolatable signals Z_{W}, Z_{E}, Z_{N}, Z_{S}, Z_{WE} and Z_{NS} to interpolation signal Z_{NEW} on the Z-axis in the range of 0<α (or β)<1, so that the lookup table stores θ₃ varying with θ₁, θ₂ and α (or β). Therefore, real-time implementation of scan conversion can be achieved by extracting θ₃ using the lookup table without calculation.

Although exemplary embodiments of the present disclosure have been described for illustrative purposes, those skilled in the art will appreciate that various modifications, additions and substitutions are possible, without departing from the essential characteristics of the disclosure. Therefore, exemplary embodiments of the present disclosure have been described for the sake of brevity and clarity. Accordingly, one of ordinary skill would understand that the scope of the disclosure is not limited by the explicitly described above embodiments but by the claims and equivalents thereof.

### INDUSTRIAL APPLICABILITY

As described above, the present disclosure is highly useful for application in the fields of real-time implementation of scan conversion by vector-interpolating four adjacent complex signals in C-mode by using a weight according to distance to prevent directional distortion of velocity data with a lookup table made for vector interpolation.

### CROSS-REFERENCE TO RELATED APPLICATION

If applicable, this application claims priority under 35 U.S.C §119(a) of Patent Application No. 10-2012-0003290, filed on Jan. 11, 2012 in Korea, the entire content of which is incorporated herein by reference. In addition, this non-provisional application claims priority in countries, other than the U.S., with the same reason based on the Korean patent application, the entire content of which is hereby incorporated by reference.

1. A scan converter for generating an interpolation signal by performing vector interpolation to represent, in Cartesian coordinates, a complex signal obtained in polar coordinates by a transducer, the scan converter comprising:
a complex signal selector configured to select four complex signals adjacent to the interpolation signal;
a first temporal interpolation signal generator configured to generate four first temporal interpolation signals by vector-interpolating every two complex signals which are two adjacent interpolatable signals among the selected four complex signals by using a weight according to a distance between each of the two adjacent interpolatable signals and the interpolation signal;
a second temporal interpolation signal generator configured to generate two second temporal interpolation signals by vector-interpolating every two first temporal interpolation signals which are two opposite interpolatable signals centering on the interpolation signal among the generated four first temporal interpolation signals by using a weight according to a distance between each of the two opposite interpolatable signals and the interpolation signal; and
an interpolation signal generator configured to generate the interpolation signal by vector-interpolating the generated two second temporal interpolation signals based on an averaged weight for averaging the generated two second temporal interpolation signals.

2. The scan converter of claim 1, wherein the complex signal selector makes selections of two scan lines as a first scan line and a second scan line adjacent to the interpolation signal among multiple scan lines in radial directions, two complex signals which are adjacent to the interpolation signal and are located on the first scan line, and two complex signals which are adjacent to the interpolation signal and are located on the second scan line.

3. The scan converter of claim 1, wherein the weight according to the distance used by the first temporal interpolation signal generator is a ratio of a distance between one of the adjacent interpolatable signals and the interpolation signal to a sum of the distance between the one interpolatable signal and the interpolation signal and a distance between the other of the adjacent interpolatable signals and the interpolation signal.

4. The scan converter of claim 1, wherein the weight according to distance used by the second temporal interpolation signal generator is a ratio of a distance between one of the opposite interpolatable signals and the interpolation signal to a sum of the distance between the one interpolatable signal and the interpolation signal and a distance between the other of the interpolatable signals and the interpolation signal.

5. The scan converter of claim 1, wherein the averaged weight is 0.5.

6. The scan converter of claim 1, wherein the interpolation signal includes distance information obtained by a geometric mean of distances of the two second temporal interpolation signals and phase information obtained by an arithmetic mean of phases of the two second temporal interpolation signals.

7. The scan converter of claim 1, further comprising:
an interpolation phase information extractor configured to extract phase information of the first and second temporal interpolation signals and of the interpolation signal from a lookup table,
wherein the lookup table stores the phase information predetermined according to phase magnitudes θ₁ and θ₂ of the two interpolatable signals and weights α and β depending on the respective distances from the two interpolatable signals to the interpolation signal.

8. The scan converter of claim 7, wherein the lookup table stores the phase information expressed by
$${θ}_{3} = α {θ}_{1} + {βθ}_{2} = {tan}^{- 1} ( \frac{sin ( α {θ}_{1} + {βθ}_{2} )}{cos ( {αθ}_{1} + {βθ}_{2} )} )$$
where θ₃ denotes the phase information and α+β=1.

9. A vector interpolation method of generating an interpolation signal by performing vector interpolation to enable a scan converter to represent, in Cartesian coordinates, a complex signal obtained in polar coordinates by a transducer, the method comprising:
selecting four complex signals adjacent to the interpolation signal;
generating four first temporal interpolation signals by vector-interpolating every two complex signals which are two adjacent interpolatable signals among the selected four complex signals by using a weight according to a distance between each of the two adjacent interpolatable signals and the interpolation signal;
generating two second temporal interpolation signals by vector-interpolating every two first temporal interpolation signals which are two opposite interpolatable signals centering on the interpolation signal among the generated four first temporal interpolation signals by using a weight according to a distance between each of the two opposite interpolatable signals and the interpolation signal; and
generating the interpolation signal by vector-interpolating the generated two second temporal interpolation signals based on an averaged weight for averaging the generated two second temporal interpolation signals.

10. The vector interpolation method of claim 9, wherein the averaged weight is 0.5.

11. The vector interpolation method of claim 9, further comprising
extracting phase information of the first and second temporal interpolation signals and of the interpolation signal from a lookup table,
wherein the lookup table stores the phase information predetermined according to phase magnitudes θ₁ and θ₂ of the two interpolatable signals and weights α and β depending on the respective distances from the two interpolatable signals to the interpolation signal.

12. An ultrasound diagnostic apparatus, comprising:
a transducer configured to convert an electric analog signal into an ultrasound signal, transmit the ultrasound signal to a subject, and convert an ultrasound signal reflected from the subject into an electric analog signal;
a beamformer configured to form a receive-focusing signal based on the converted electric analog signal;
a demodulator configured to form I data comprising in-phase components and Q data comprising quadrature-phase components by demodulating the receive-focusing signal into a baseband signal; and
a scan converter configured to generate an interpolation signal by performing vector interpolation to represent, in Cartesian coordinates, a complex signal obtained in polar coordinates, based on the I data and the Q data,
wherein the scan converter generates the interpolation signal by vector-interpolating four complex signals adjacent to the interpolation signal by using a weight according to a distance between each of the four complex signals and the interpolation signal.

13. The ultrasound diagnostic apparatus of claim 12, further comprising:
an analog/digital (A/D) converter configured to convert the electric analog signal transformed by the transducer into an electric digital signal.

14. The ultrasound diagnostic apparatus of claim 12, wherein the scan converter comprises:
a complex signal selector configured to select the four complex signals adjacent to the interpolation signal;
a first temporal interpolation signal generator configured to generate four first temporal interpolation signals by vector-interpolating every two complex signals which are two adjacent interpolatable signals among the selected four complex signals by using a weight according to a distance between each of the two adjacent interpolatable signals and the interpolation signal;
a second temporal interpolation signal generator configured to generate two second temporal interpolation signals by vector-interpolating every two first temporal interpolation signals which are two opposite interpolatable signals centering on the interpolation signal among the generated four first temporal interpolation signals by using a weight according to a distance between each of the two opposite interpolatable signals and the interpolation signal; and
an interpolation signal generator configured to generate the interpolation signal by vector-interpolating the generated two second temporal interpolation signals based on an averaged weight for averaging the generated two second temporal interpolation signals.

15. The ultrasound diagnostic apparatus of claim 14, wherein the averaged weight is 0.5.

16. The ultrasound diagnostic apparatus of claim 14, wherein the scan converter further comprises an interpolation phase information extractor configured to extract phase information of the first and second temporal interpolation signals and of the interpolation signal from a lookup table, wherein the lookup table stores the phase information predetermined according to phase magnitudes θ₁ and θ₂ of the two interpolatable signals and weights α and β depending on the respective distances from the interpolatable signals to the interpolation signal.
